(19) United States
(12) Patent Application Publication
(10) Pub. No.: US 2003/0153914 A1
(43) Pub. Date: Aug. 14, 2003

(54) Rod distance retainer

(75) Inventors: Kazuya Oribe, Tokyo (JP); Hiroshi Takamido, Nagoya-shi (JP)

Correspondence Address: GREENBLUM & BERNSTEIN, P.L.C., 1950 ROLAND CLARKE PLACE, RESTON, VA, 20191, US

(73) Assignee: SHOWA IKA KOHGYO CO., LTD., Aichi (JP)

(21) Appl. No.: 10/359,174

(22) Filed: Feb. 6, 2003

(30) Foreign Application Priority Data
Feb. 8, 2002 (JP) JP2002-033040

(51) Int. Cl. A61B 17/56

(52) U.S. Cl. 606/61

(57) ABSTRACT

A rod distance retainer comprises a coupler link including rod engaging concave portions which engage a pair of rods fixing a plurality of adjacent vertebral bodies, and fixing screws which fix the rods which is engaged with the rod engaging concave portions. In the rod distance retainer of the present invention, taper faces are formed on tip portions of the fixing screws to enable the fixing screws to abut on peripheral surfaces of the rods.

### BACKGROUND OF THE INVENTION

1. Field of the Invention

The present invention relates to a rod distance retainer for retaining a pair of rods which are fixed appropriately onto a plurality of vertebral bodies so as to constantly retain a distance between the plurality of vertebral bodies being spaced appropriately. More specifically, the present invention relates to a rod distance retainer which is easy to fabricate, fit to the rods, and accommodate to inclination of the rods.

2. Description of the Related Art

A vertebral body retainer 2 shown in FIGS. 1 and 2 is known as the related art for retaining vertebral bodies which are appropriately spaced. The vertebral body retainer 2 includes a pair of rods 5A and 5B, a plurality of implants 3, and a coupler link 7. Both end portions of the pair of rods 5A and 5B are fixed to a plurality of vertebral bodies 1, which are appropriately spaced, by use of the plurality of implants 3 such as screws. Thereby, a distance between the pair of vertebral bodies 1 is retained constantly. Moreover, a constitution of coupling the rods 5A and 5B with the coupler link 7 is adopted so as to constantly retain the space between the pair of rods 5A and 5B.

FIG. 2 shows a structure of the coupling link 7 of the vertebral body retainer 2. Specifically, the coupler link 7 includes a fixed link 9 provided with a semicircular engaging concave portion 9A on a tip portion thereof so as to be engageable with the rod 5A, and a movable link 11 provided with a semicircular engaging concave portion 11A on a tip portion thereof so as to be engageable with the rod 5B. Moreover, the fixed link 9 and the movable link 11 are retractably coupled together.

Specifically, an inserting rod 13 with a quadrangular cross section formed on a base end side of the movable link 11 is movably inserted into an engaging hole (not shown) with a quadrangular cross section formed as an opening on a base end side of the fixed link 9. Then, the inserting rod 13 is fixed by a fixing screw 15 being screwed into the fixed link 9. Therefore, it is possible to coordinate the engaging concave portions 9A and 11A of the both links 9 and 11 with the distance between the both rods 5A and 5B in the state of loosening the fixing screw 15.

To fix the rods 5A and 5B to the engaging concave portions 9A and 11A, tapped holes 17 and 19 for allowing insertion of fixing screws (not shown) are formed on the both links 9 and 11, respectively so as to communicate with the engaging concave portions 9A and 11A.

### SUMMARY OF THE INVENTION

However, in the coupler link 7 proposed in the foregoing description, diameters of the engaging concave portions 9A and 11A are formed substantially equal to diameters of the rods 5A and 5B, and the movable link 11 is not rotatable with respect to the fixed link 9. Accordingly, if any one of the rods 5A and 5B is slightly inclined, there is a problem that the coupler link 7 is difficult to deal with such inclination.

Moreover, since the tapped holes 17 and 19 are formed obliquely against upper surfaces of the fixed link 9 and the movable link 11, formation of the tapped holes 17 and 19 becomes complicated and the fixing screws need to be rotated in oblique directions. Thus, the coupler link 7 has a problem in terms of operability. Furthermore, the fixing screws to be screwed into the tapped holes 17 and 19 have structures to press the rods 5A and 5B by flat faces on tips thereof from orthogonal directions with respect to shaft centers of the rods 5A and 5B, therefore the fixing screws constitute line contacts. In this regard, an improvement for tighter fixation is requested.

The present invention has been made in consideration of the above problems. It is an object of the present invention to provide a rod distance retainer, which is capable of tightly fixing a pair of rods that are fixed to vertebral bodies and is excellent in operability and workability.

To achieve the object described above, the present invention provides a rod distance retainer, comprising: a coupler link including rod engaging concave portions which engages a pair of rods, the rods fixing a plurality of adjacent vertebral bodies; and fixing screws which fix the rods which is engaged with the rod engaging concave portions, wherein taper faces are formed on tip portions of the fixing screws to enable the fixing screws to abut on peripheral surfaces of the rods.

### BRIEF DESCRIPTION OF THE DRAWINGS

The invention will now be described with reference to the accompany drawings wherein;

FIG. 1 is a schematic view showing a structure of a proposed a vertebral body retainer;

FIG. 2 is a perspective view showing a structure of a proposed rod distance retainer;

FIG. 3 is a schematic view showing a structure of a vertebral body retainer according to an embodiment of the present invention;

FIG. 4A is a top plan view showing a structure of a rod distance retainer according to an embodiment of the present invention; and

FIG. 4B is a cross-sectional view showing a structure of a rod distance retainer according to an embodiment of the present invention.

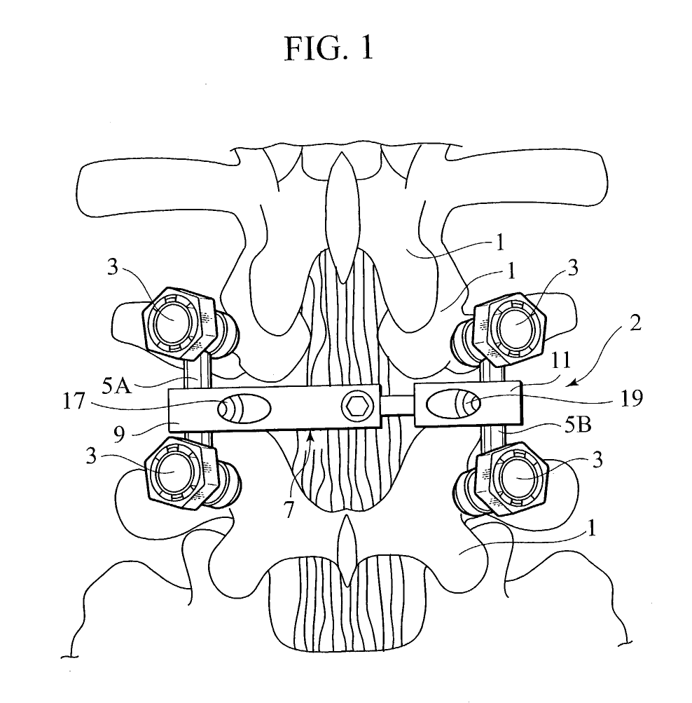

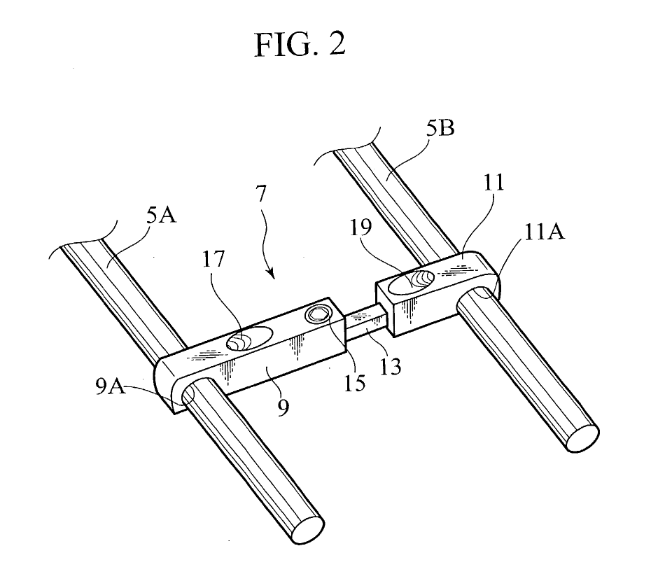

### DETAILED DESCRIPTION OF THE PREFERRED EMBODIMENT

Hereinafter, description will be made of embodiments of the present invention with reference to the drawings.

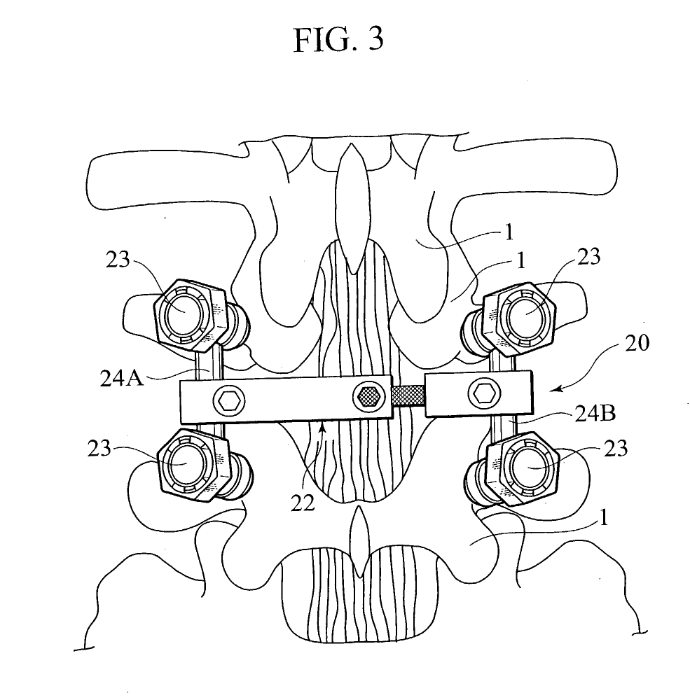

FIG. 3 shows a vertebral body retainer 20 using a rod distance retainer according to an embodiment of the present invention. The vertebral body retainer 20 mainly includes a pair of rods 24A and 24B, a plurality of implants 23, and a coupler link 22. Both end portions of the pair of rods 24A and 24B are fixed to a plurality of vertebral bodies 1, which are appropriately spaced, by use of the plurality of implants 23 such as screws, and a distance between the pair of vertebral bodies 1 is thereby retained constantly. Moreover, a constitution of coupling the rods 24A and 24B with the coupler link 22 is adopted so as to constantly retain the space between the pair of rods 24A and 24B.

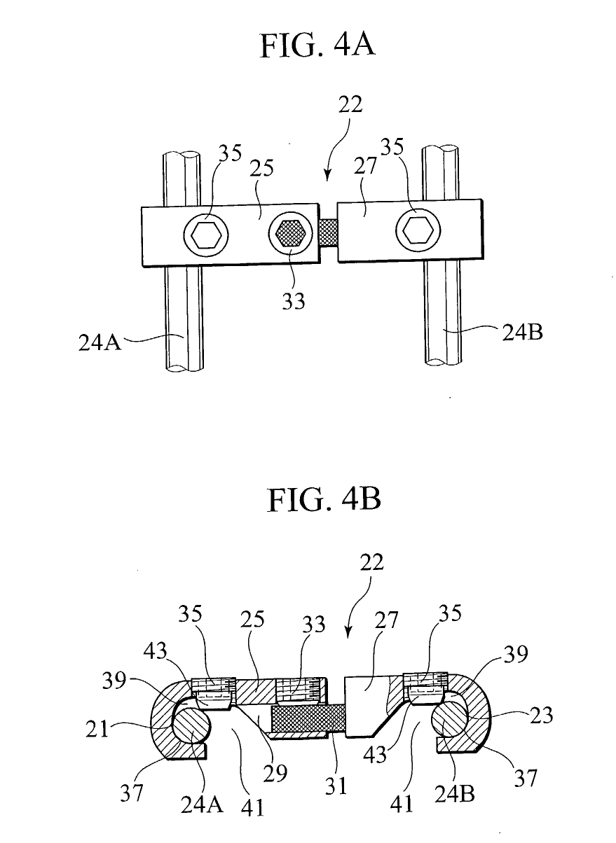

FIGS. 4A and 4B shows a constitution of the rod distance retainer, which comprises the coupler link 22. The coupler link 22 is constituted by rod engaging concave portions 21 and 23 being provided on both ends of the coupler link 22, a fixed link 25 provided with the rod engaging concave portion 21 on a tip portion thereof, and a movable link 27 provided with the rod engaging concave portion 23 on a tip portion thereof. A columnar inserting rod 31 provided on a base end portion of the movable link 27 is movably and rotatably fitted into an engaging hole 29 which is formed along the longitudinal direction on the fixed link 25. Moreover, the inserting rod 31 is fixed by a fixing screw 33 which is screwed into the fixed link 25.

Therefore, when fixation of the inserting rod 31 by the fixing screw 33 is released, the inserting rod 31 can be moved in the axial direction along the engaging hole 29. Accordingly, it is possible to coordinate a distance between the rod engaging concave portions 21 and 23 with a distance between the pair of rods 24A and 24B and also to rotate the movable link 27 with respect to the fixed link 25. Hence, if the rod 24B is slightly inclined against the rod 24A, it is possible to engage the rod engaging concave portions 21 and 23 to the both rods 24A and 24B respectively in an aligned state.

The engaging concave portions 21 and 23 are formed symmetrically. Each of the engaging concave portions 21 and 23 includes an arc-shaped support 37 for supporting the rod 24A or 24B when a fixing screw 35 for rod-fixing is screwed into the fixed link 25 or the movable link 27 so as to fix the rod 24A or 24B by pressure. Moreover, each of the engaging concave portions 21 and 23 includes a clearance 39 on an upper part of the support 37 for allowing upward movement of the rod 24A or 24B in the event of loosening the fixing screw 35. Furthermore, apertures 41 are formed obliquely toward inner lower sides, so as to communicate with the rod engaging concave portions 21 and 23, respectively.

In other words, each of the rod engaging concave portions 21 and 23 is formed as a long hole along the moving direction of the fixing screw 35, i.e. in the vertical direction in FIG. 4B, and is arranged to communicate with the aperture 41 on one side of this long hole.

When the rods 24A and 24B are engaged with the rod engaging concave portions 21 and 23, each of the fixing screw 35 is located in a position which is shifted inward from the shaft center of the rod 24A or 24B, i.e. toward the aperture 41 side. Moreover, the fixing screws 35 are screwed into tapped holes, which are formed in the orthogonal direction to upper surfaces of the fixed link 25 and the movable link 27 respectively, at communicating portions of the rod engaging concave portions 21 and 23 with the apertures 41. Furthermore, a tip portion of the fixing screw 35 is formed into a tapered shape having a taper face 43, so as to be capable of abutting on the outer periphery of the rod 24A or 24B which is located in the rod engaging concave portion 21 or 23.

In the vertebral body retainer 20 of the above-described constitution, as shown in FIGS. 4A and 4B, the rod engaging concave portions 21 and 23 provided on the both ends of the coupler link 22 are engaged with the pair of rods 24A and 24B, which are integrated with the vertebral bodies 1 by use of the plurality of implants 23. Here, even if the rod 24B is slightly inclined against the rod 24A, it is possible to engage the both rods easily in response to such inclination because the clearances 39 are provided on the rod engaging concave portions 21 and 23, and because the movable link 27 is arranged as rotatable relatively to the fixed link 25.

The distance between the rod engaging concave portions 21 and 23 is adjusted in response to the distance between the pair of rods 24A and 24B. Thereafter, the inserting rod 31 is fixed by the fixing screw 33 so as to integrate the fixed link 25 with the movable link 27, and then the fixing screws 35 are tighten up. In this way, the rods 24A and 24B can be surely fixed into the rod engaging concave portions 21 and 23.

When the fixing screws 35 are tightened up, the taper faces 43 provided on the tip portions of the fixing screws 35 abut on the outer peripheries of the rods 24A and 24B respectively, and act to press the rods 24A and 24B obliquely downward. Accordingly, the rods 24A and 24B can be surely fixed by pressure into inner peripheries of the rod engaging concave portions 21 and 23.

In this event, since the taper face 43 of the fixing screw 35 contacts with the outer periphery of the rod 24A or 24B, large pressure is generated by a wedge effect, and the contact position constitutes a point contact. Accordingly, the contact portion will receive substantial stress. Hence, the contact portion is elastically deformed and is prevented from loosening. Meanwhile, reactive force acts on the tip portion of the fixing screw 35 upon tightening, so as to slant the fixing screw 35 against the tapped hole. Accordingly, the fixing screws 35 are prevented from loosening inside the tapped holes, whereby the rods 5A and 5B can be surely and tightly fixed.

According to the rod distance retainer of this embodiment, the taper face provided on the tip portion of the fixing screw is allowed to abut on the outer periphery of the rod so as to fix the rod by pressure to the rod engaging concave portion. Therefore, the rod can be surely and tightly fixed by pressure. As a consequence, the vertebral bodies which are engaged with the rod can be surely fixed. Moreover, the workability is improved because the tapped holes on the coupler link do not have to be formed obliquely. In addition, the operability is also improved because the fixing screws do not have to be rotated in the oblique directions.

The entire content of a Japanese Patent Application No. P2002-33040 with a filing date of Feb. 8, 2002 is herein incorporated by reference.

Although the invention has been described above by reference to certain embodiments of the invention, the invention is not limited to the embodiments described above will occur to these skilled in the art, in light of the teachings. The scope of the invention is defined with reference to the following claims.

What is claimed is:

1. A rod distance retainer, comprising:
a coupler link including rod engaging concave portions which engage a pair of rods, the rods fixing a plurality of adjacent vertebral bodies; and
fixing screws which fix the rods to the rod engaging concave portions,
wherein taper faces are formed on tip portions of the fixing screws to enable the fixing screws to abut on peripheral surfaces of the rods.

2. The rod distance retainer of claim 1,
wherein the fixing screw is located in a position being shifted from a shaft center of the rod engaged with the rod engaging concave portion toward an aperture of the rod engaging concave portion.

3. The rod distance retainer of claim 1, further comprising:
clearances provided on the rod engaging concave portions respectively, the clearances being configured to allow movement of the rods in an inserting/pulling direction of the fixing screw.

4. The rod distance retainer of claim 1,
wherein the coupler link includes a fixed link and a movable link, and
the movable link is detachably and rotatably fitted into the fixed link.

5. The rod distance retainer of claim 1,
wherein the fixing screw is screwed in an orthogonal direction with respect to an upper surface of the coupler link.
